(19) United States
(12) Patent Application Publication
(10) Pub. No.: US 2021/0260320 A1
(43) Pub. Date: Aug. 26, 2021

(54) INTUBATING ENDOSCOPIC DEVICE

(71) Applicant: Andrew Maslow, Needham, MA (US)

(72) Inventor: Andrew Maslow, Needham, MA (US)

(21) Appl. No.: 17/065,250

(22) Filed: Oct. 7, 2020

Related U.S. Application Data
(63) Continuation of application No. 16/368,377, filed on Mar. 28, 2019, now abandoned.
Application No. 16/368,377 is a continuation-in-part of application No. 15/717,351, filed on Sep. 27, 2017.
(60) Provisional application No. 62/649,187, filed on Mar. 28, 2018.
(60) Provisional application No. 62/400,340, filed on Sep. 27, 2016.

(51) Int. Cl. A61M 16/04; A61B 1/267; A61B 1/005; A61B 1/012; A61B 1/06; A61B 1/01; A61B 1/015; A61B 1/07

(52) U.S. Cl. CPC A61M 16/0488; A61M 2205/586; A61B 1/0052; A61B 1/0125; A61B 1/0661; A61B 1/01; A61M 16/0418; A61B 1/015; A61B 1/0051; A61M 16/0486; A61M 16/0484; A61B 1/07; A61M 2205/0216; A61M 16/0463; A61M 16/0461; A61B 1/267

(57) ABSTRACT

An intubating device is provided for use with an endotracheal tube. In some embodiments, the intubating device includes a sheath having a distal end and a proximal end, and the sheath is configured to be introduced into a body cavity. The sheath has a rigid section, and the sheath has a flexible section at the distal end. The flexible section is selectively bendable. A control housing is located at the proximal end. The control housing includes a deflector for selectively bending the flexible section of the sheath. In some embodiments, an intubating device includes a sheath having a malleable first section and a second section that is selectively bendable located at a distal end of the sheath.

### CROSS-REFERENCE TO RELATED APPLICATIONS

This application is a continuation of U.S. patent application Ser. No. 16/368,377 titled “INTUBATING ENDOSCOPIC DEVICE” filed Mar. 28, 2019, which claims the benefit under 35 U.S.C. § 119(e) to U.S. Provisional Application Ser. No. 62/649,187 titled “INTUBATING ENDOSCOPIC DEVICE,” filed Mar. 28, 2018, and which is a continuation-in-part of U.S. patent application Ser. No. 15/717,351 titled “INTUBATING ENDOSCOPIC DEVICE” filed Sep. 27, 2017, which claims the benefit under 35 U.S.C. § 119(e) to U.S. Provisional Application Ser. No. 62/400,340 titled “INTUBATING ENDOSCOPIC DEVICE,” filed Sep. 27, 2016, each of which is incorporated by reference herein in its entirety for all purposes.

### FIELD OF THE DISCLOSURE

The present disclosure is related to an intubating device. The present disclosure is particularly related to an intubating device for assisting in introducing an endotracheal tube into an airway of a patient.

### SUMMARY OF THE DISCLOSURE

In one aspect of the present disclosure, an intubating device is provided for use with an endotracheal tube. The intubating device includes a sheath having a distal end and a proximal end, the sheath being configured to be introduced into a body cavity, the sheath having a rigid section, and the sheath having a flexible section at the distal end, the flexible section being selectively bendable; and a control housing at the proximal end, the control housing comprising a deflector for selectively bending the flexible section of the sheath.

In some embodiments, the sheath extends along any or all of a light source channel, an imaging channel, and a gas channel.

In some embodiments, the sheath is configured and dimensioned to be received within an endotracheal tube having an inner diameter of at least 6 mm.

In some embodiments, the rigid section is between 30 centimeters and 32 centimeters in length, and the flexible section is between 3 centimeters and 5 centimeters in length.

In some embodiments, the rigid section comprises a curved portion towards the distal end adjacent to the flexible section.

In some embodiments, the rigid section has a centerline along its length that is nonlinear.

In some embodiments, any or all of the imaging channel, the gas channel, and the light source channel terminate at the distal end of the sheath.

In some embodiments, the imaging channel is configured to be connected to an optical viewer.

In some embodiments, the gas channel is configured to be connected to at least one of a gas source and a suction source.

In some embodiments, the device includes a tube stop configured to secure a first end of an endotracheal tube to the intubating device.

In some embodiments, the device includes a fluid (saline) channel.

In some embodiments, the device includes at least one flexible wire extending along the length of the sheath and connected to the deflector.

Another aspect of the present disclosure is directed to an intubating device for use with an endotracheal tube, the intubating device including a sheath having a distal end and a proximal end, the sheath being configured to be introduced into a body cavity, the sheath having a rigid section, and the sheath having a flexible section at the distal end, the flexible section being selectively bendable; the sheath extending along a light source channel, an imaging channel, and a gas channel; a control housing at the proximal end; the control housing comprising a deflector for selectively bending the flexible section of the sheath; wherein the sheath is configured to be received within an endotracheal tube having an inner diameter of at least 6 mm; and wherein the rigid section is between 30 centimeters and 32 centimeters in length, and the flexible section is between 3 centimeters and 5 centimeters in length.

Another aspect of the present disclosure is directed to an intubating device for use with an endotracheal tube, the intubating device comprising a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and a control housing at the proximal end; the control housing comprising a deflector for selectively bending the second section of the sheath.

In some embodiments, the sheath is configured to be received within an endotracheal tube having an inner diameter of at least 6 mm. In some embodiments, wherein the first section is 22 centimeters in length, and the second section is 2 centimeters in length.

In some embodiments, the intubating device further comprises any or all of a light source channel, an imaging channel, and a gas channel enclosed by the sheath.

In some embodiments, the intubating device further comprises at least one flexible wire extending along the length of the sheath connected to the deflector and to the second section. In some embodiments, the at least one flexible wire comprises two wires.

In some embodiments, the first section is 22 centimeters in length, and the second section is 2 centimeters in length.

In some embodiments, the first section comprises a curved portion towards the distal end adjacent to the second section.

In some embodiments, the intubating device further comprises any or all of a light source channel, an imaging channel, and a gas channel enclosed by the sheath.

In some embodiments, the imaging channel, the gas channel, and the light source channel terminate at the distal end of the sheath.

In some embodiments, the imaging channel is configured to be connected to an optical viewer.

In some embodiments, the gas channel is configured to be connected to at least one of a gas source and a suction source.

In some embodiments, the intubating device further comprises a fluid channel.

In some embodiments, the intubating device further comprises a tube stop configured to secure a first end of an endotracheal tube to the intubating device.

In some embodiments, the intubating device further comprises at least one flexible wire extending along the length of the sheath connected to the deflector and to the second section. In some embodiment, the at least one flexible wire comprises two wires.

According to another aspect of the present disclosure, an intubating device for use with an endotracheal tube is provided, and the intubating device comprises a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and the sheath extending along a light source channel, an imaging channel, and a gas channel; a control housing at the proximal end; the control housing comprising a deflector for selectively bending the second section of the sheath; wherein the sheath is configured to be received within an endotracheal tube having an inner diameter of at least 6 mm; and wherein the first section is between 30 centimeters and 32 centimeters in length, and the second section is between 3 centimeters and 5 centimeters in length.

According to another aspect of the present disclosure, an intubating kit for use with an endotracheal tube is provided, and the kit comprises a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and a control housing at the proximal end; the control housing comprising a deflector for selectively bending the second section of the sheath; and a shaping tool configured to apply a curvature to the first section of the intubating device, so the curvature of the first section allows the first section to extend through a nasal cavity and pharyngeal passages of a patient.

In some embodiments, the first section of the intubating device is made of a first material having a first modulus of elasticity and the shaping tool is made of a second material having a second modulus of elasticity, the second material being than the first material, the first modulus of elasticity being greater than the second modulus of elasticity.

In some embodiments, the shaping tool includes a U-shaped wall for applying the curvature of the U-shaped wall to the first section.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A shows a partially exploded view of a first embodiment of the intubating device of the present disclosure;

FIG. 1B shows the flexible portion of the device of FIG. 1A at various curvatures;

FIG. 2 shows a view of the distal end thereof;

FIG. 3A shows the embodiment of FIG. 1A being inserted into an airway;

FIG. 3B shows how the position of the embodiment of FIG. 1A can be adjusted as it is inserted into an airway;

FIG. 4A shows a partially exploded view of a second embodiment of the intubating device of the present disclosure;

FIG. 4B shows the flexible portion of the device of FIG. 4A at various curvatures;

FIG. 5 shows a view of the distal end thereof;

FIG. 6 shows the embodiment of FIG. 4A being inserted into an airway;

FIG. 7A shows the deflector in a first position;

FIG. 7B shows the deflector in a second position;

FIG. 7C shows the deflector in a third position;

FIG. 8A shows the flexible section of a sheath bent to a first geometry;

FIG. 8B shows the flexible section of a sheath bent to a second geometry;

FIG. 8C shows the flexible section of a sheath bent to a third geometry; and

FIG. 8D shows the flexible section of a sheath bent to a fourth geometry;

FIG. 9 shows a schematic sectional view of a patient with an embodiment of an intubating device used for nasal intubation of the patient;

FIG. 10 shows a sectional view of a patient with an embodiment of an intubating device 1001 used for nasal intubation of the patient;

FIG. 11 shows an embodiment of a shaping tool that can be used to alter the shape of the rigid section of a sheath of an intubating device; and

FIG. 12 shows an embodiment of a shaping tool that can be used to alter the shape of the rigid section of a sheath of an intubating device.

### DETAILED DESCRIPTION OF THE DISCLOSURE

The present disclosure relates generally to an intubating endoscopic device. Particularly, in some embodiments the present disclosure relates to a bronchoscope having a rigid section along the majority of its length and a flexible section at its distal end.

When not performed properly, tracheal intubation can cause respiratory-related injuries. Maintaining a patient's airway is important for a patient undergoing any procedure, particularly when the patient is under general anesthesia. If an airway cannot be quickly established, prolonged hypoxia can result, risking damage to vital organs such as the heart and the brain.

Traditional laryngoscopes lift the mandible and the tongue anteriorly (upward), which not only widens the visual space but also helps to lift the epiglottis up to allow visualization of the vocal cords and visualization of where to place the endotracheal tube (ETT) alongside it. This procedure is associated with significant cervical spine motion.

Flexible fiberoptic scopes require two hands and do not lift the mandible and adjacent soft tissues, but instead ‘snake around’ structures until the airway is seen. Accessory devices or aids might include a special oral airway to maintain a mid-line position and guide the flexible scope around the tongue and into the pharynx (i.e. past the base of the tongue. Another ‘aid’ is to have a second person either pull the tongue forward or manually lift the mandible forward. This procedure is associated with significantly less or no cervical spine motion compared to traditional laryngoscopes.

Videolaryngoscopes are like traditional laryngoscopes, but use a visual scope at its tip (rather than the user's own eyes) and, while the user holds the videolaryngoscope with one hand, the endotracheal tube with a special rigid stylet is passed alongside the videolaryngoscope into the airway with the user's other hand. This may require less mandibular displacement (i.e. jaw lift or thrust) as the vocal cords are seen at the tip of the device. This procedure is associated with less cervical spine motion than traditional laryngoscopy.

Traditional laryngoscopes and videolaryngoscopes are not low profile devices, and require enough space to pass both the device and, alongside the device, the styletted laryngoscope.

Flexible fiberoptic scopes and the intubating endoscopic device of the present disclosure are lower profile devices that only require the one device (with ETT on it) passed into the patient's mouth, behind the tongue and then guided toward the airway.

According to some embodiments, the device of the present disclosure has a straight rigid portion and a distal flexible portion, such that the rigid portion allows the user to lift the tongue and soft tissues. The flexible portion at the tip allows the user to search for and steer toward the airway structure (such as the vocal cords). Like a flexible bronchoscope this procedure is associated with less or no cervical spine motion compared to traditional laryngoscopy.

Embodiments of the device of the present disclosure are low profile, easy to handle scope that has the benefits of being rigid to allow easier handling using a single hand to place, and the ability to lift the tongue and mandible to expose the larynx, while having a flexible tip to allow search for and steering toward the vocal cords.

In contrast, a flexible fiberoptic scope requires two hands to guide the flexible fiberoptic scope.

Because a flexible fiberoptic scope is passed through the vocal cords into the distal part of the trachea to guide the ETT, this might cause trauma to the distal airway tissues. Unlike a flexible scope, the device of the present disclosure does not have to pass all the way into the airway or can even sit above the airway and guide) the ETT into the trachea (and allow a user to watch the ETT pass into the trachea). Unlike laryngoscopes the device of the present disclosure can pass slightly into the airway to provide a more direct guide of the ETT. The device of the present disclosure can exhibit advantages of a flexible scope and a laryngoscope.

As discussed herein, the intubating device of the present disclosure includes a rigid section to allow for increased user accuracy in placement of an endotracheal tube, and also includes a flexible section to allow the placement of the endotracheal tube to adjust to the anatomy of a user's pharynx while minimizing potential blunt trauma injury to the airway structures.

Thus, some aspects and advantages of the intubating device of the present disclosure are that the intubating device can be grasped by one hand of a user during use. In particular, the intubating device can be held by a user in one hand to secure an airway, without the use of a second device, such as a laryngoscope blade.

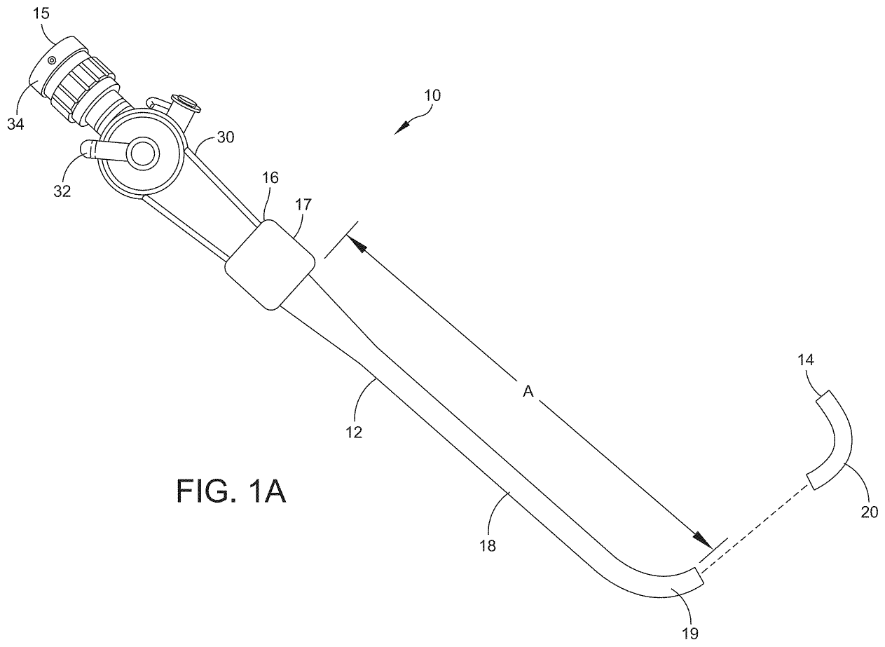

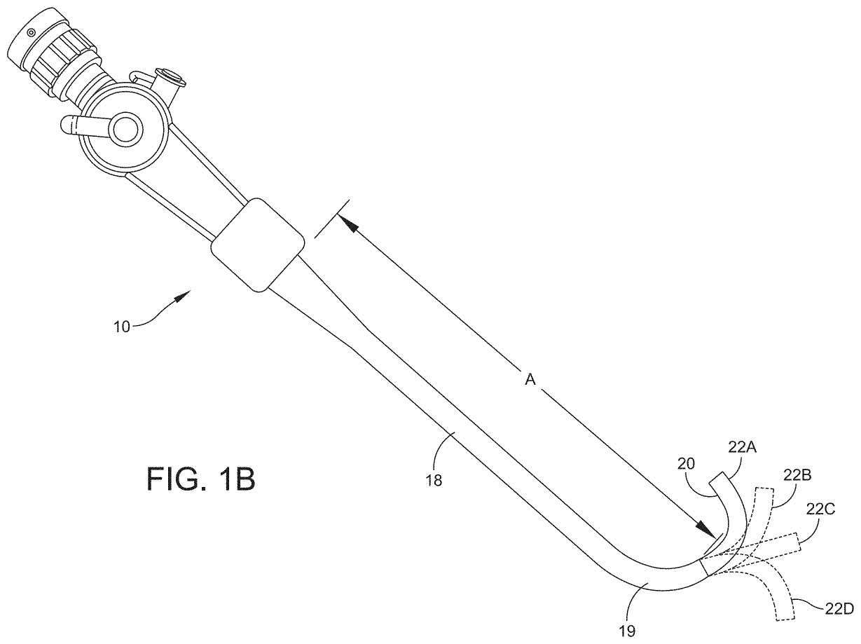

Other aspects and advantages of the intubating device of the present disclosure are, for example: it can conform to the configuration of the pharynx of a patient; it is portable; it is low profile (small, not long) easy to handle and to use and therefore provides for users to quickly become expert users of the intubating device; it is adjustable; it can be made inexpensively; it is generally straight or can be slightly bent, has a flexible and maneuverable tip, and is durable. FIG. 1A shows a first embodiment of an intubating device 10 of the present disclosure. The intubating device 10 includes a sheath 12 that has a distal end 14 and a proximal end 16, and is configured to be introduced into a body cavity, such as a mouth of a human patient. In some uses, the intubating device can be introduced at another point along an airway of a patient. The sheath 12 of FIG. 1A is shown in a partially exploded view, having a rigid section 18 and a flexible section 20 adjacent to the rigid section 18 and at the distal end 14 of the sheath 12. Although the sheath 12 of FIG. 1A is shown in a partially exploded view, the flexible section 20 is secured to the rigid section 18 when the intubating device 10 is in use, as shown in FIG. 1B.

The rigid section 18 may be reinforced by a rigid layer, such as a metal layer. The metal layer can be a strip along the length of the rigid section 18. In some embodiments, the metal layer is a strip that extends along the length of one or more sides of the rigid section 18. In some embodiments, the metal layer encircles the rigid section 18 and extends along the length of the rigid section 18.

The flexible section 20 is configured to be selectively bendable, as shown in FIG. 1B, where the flexible section 20 has a curvature 22A that can be adjusted to various curvatures 22B, 22C, 22D of the flexible section 20 (as shown in broken lines for illustration purposes in FIG. 1B). With this arrangement, a user can adjust the curvature of the flexible section 20 of the sheath to navigate a body cavity, such as an airway of a patient.

Generally, the sheath is configured to be inserted into an airway. Generally, the sheath is configured to contain at least one light bundle in at least one light source channel and an image transmission bundle in an imaging channel. Each light bundle and each transmission bundle can be configured as a glass bundle. These glass bundles (the light bundle and the image transmission bundle) run the length of the endoscope containing thousands of glass fibers, which are for example approximately 8 microns to 10 microns in diameter. The image transmission bundle provides for light containing an image to travel along the length of the fiber, being internally reflected along its length to the eyepiece whilst still retaining a sharp image of the object. Because many of these glass fibers are bundled together, the image is built as a composite of that obtained by each fiber, similar to pixels on a television screen or another display. In some embodiments, a typical fiber optic bundle contains 10,000 glass fibers.

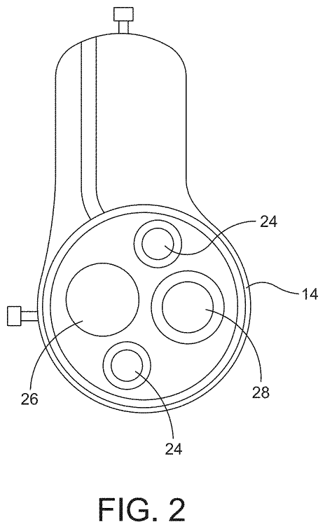

In the embodiment of FIG. 1A, the sheath 12 surrounds a light source channel 24, an imaging channel 26, and a gas channel 28, as shown in the end view of FIG. 2. A light source port is disposed at the end of the light source channel 24, and a light bundle extends within the light source channel 24. A fiber optic port is disposed at the end of the imaging channel 26, and an image transmission bundle extends within the imaging channel 26. The imaging channel 26 may be a fiber optic guide channel or a channel for another imaging medium.

Some embodiments can include two light source channels 24 as shown in FIG. 2. Each light source channel is useful for transmitting light from a light source and providing light to a patient's airway at the distal end 14 of the sheath 12. The light source for a video bronchoscope is generally halogen or incandescent, and is transmitted via fiberoptic bundles. It is appreciated that the light source may also be LED or any other light source that is customary. The light source can be a portable battery powered source or connected via a cable.

Thus aspects and advantages of the intubating device of the present disclosure include that it can be used to provide a bright light source to a patient without risking heat injury to the patient. For example, in some embodiments, the light source can be remote from the distal end of the intubating device, so there is no risk of heat injury to the patient due to the light source.

The sheath also encloses a working channel (such as a gas channel) 28, which is provided for suction, instillation of local anesthetic, and oxygen delivery to the patient's airway. The sheath is configured with an oxygen port insufflation/suction port as shown at an end of the gas channel 28.

In some embodiments, an additional biopsy/suction channel can also be provided. Such a channel can be useful for applying suction to the patient's airway at the distal end of the sheath to collect liquids and/or solids within the airway, and can be provided separately from a gas channel for providing oxygen.

In some embodiments, a fluid channel is provided. For example, in some embodiments, a fluid channel is useful for introducing fluid to the patient's airway adjacent to the distal end of the sheath. In some embodiments, the fluid channel can be used to provide saline or a fluid that includes saline. In some embodiments, the fluid channel runs the length of the endoscope.

In some embodiments, the fluid channel, the biopsy/suction channel, and/or the second light source channel 24 can be enclosed within the sheath.

In the embodiment of the intubating device 10 of FIG. 1A, the sheath 12 encloses at least one light source channel 24, the imaging channel 26, and the gas channel 28 extending along the length rigid section and flexible section of the sheath. In the embodiment of the intubating device 10 of FIG. 1A, the imaging channel 26, the gas channel 28, and the light source channels 24 terminate at the distal end 14 of the sheath 12.

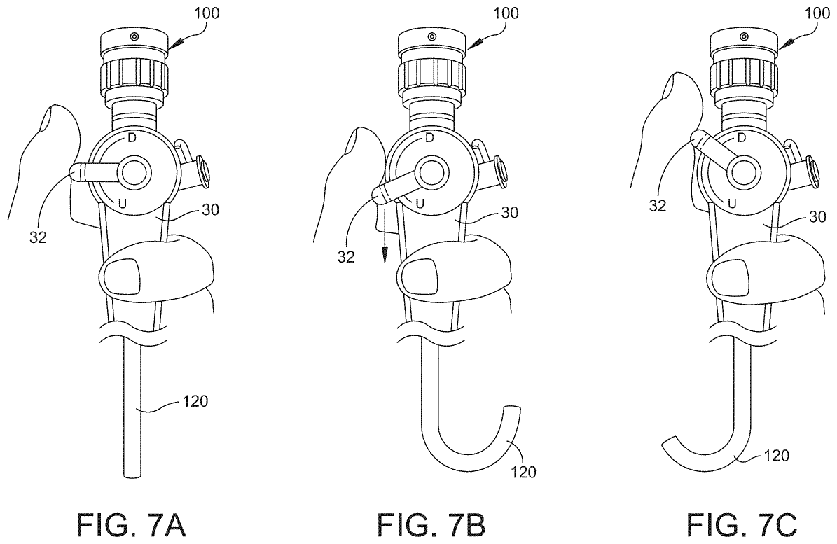

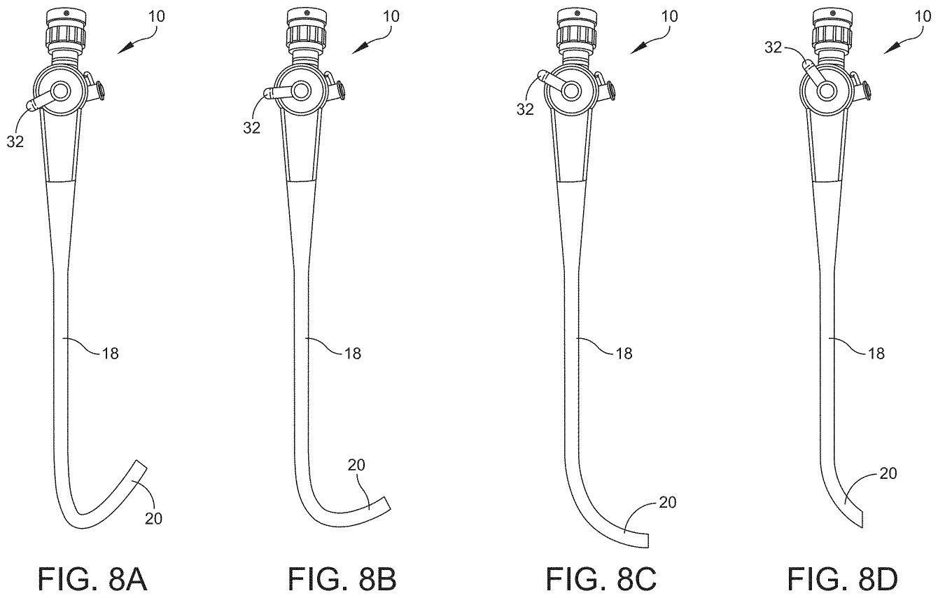

The intubating device 10 allows the user to control the curvature of the flexible section 20 of the sheath 12 with a deflector 32. The sheath 12 also encloses at least one tip bending guide wire extending along its length (not illustrated). A control housing 30 is shown adjacent to the proximal end 16 of the sheath 12 in FIG. 1A. The control housing 30 includes the deflector 32 for selectively bending the flexible section 20 of the sheath 12. The operation of the tip bending guide wire(s) and the deflector 32 is discussed in further detail below in relation to FIGS. 7A-8D.

The sheath 12 of the intubating device 10 is dimensioned and configured to be received within an endotracheal tube having an inner diameter that is at least 6 millimeters, for example between 6 millimeters and 9 millimeters. For example, the outer diameter of the rigid section 18 and flexible section 20 of the sheath can be between 5 millimeters and 6 millimeters in some embodiments. In some embodiments, the outer diameter of the rigid section 18 and the flexible section 20 of the sheath is 5 millimeters. It is also appreciated that the outer diameter of the sheath can be any dimension that is useful in intubating an airway.

In one embodiment of the device, the rigid section 18 has a length A that is between 30 centimeters and 32 centimeters in length, and the flexible section 20 that is between 3 centimeters and 5 centimeters in length when straightened.

In one embodiment of the intubating device of the present disclosure, the total length of the endoscopic device is typically in the range of 500 millimeters to 650 millimeters in length. In some embodiments, the length is 600 millimeters in length. It is appreciated that the length of the endoscopic device can be dimensioned to suit the needs of a patient having a longer or shorter airway without departing from the scope of the present invention.

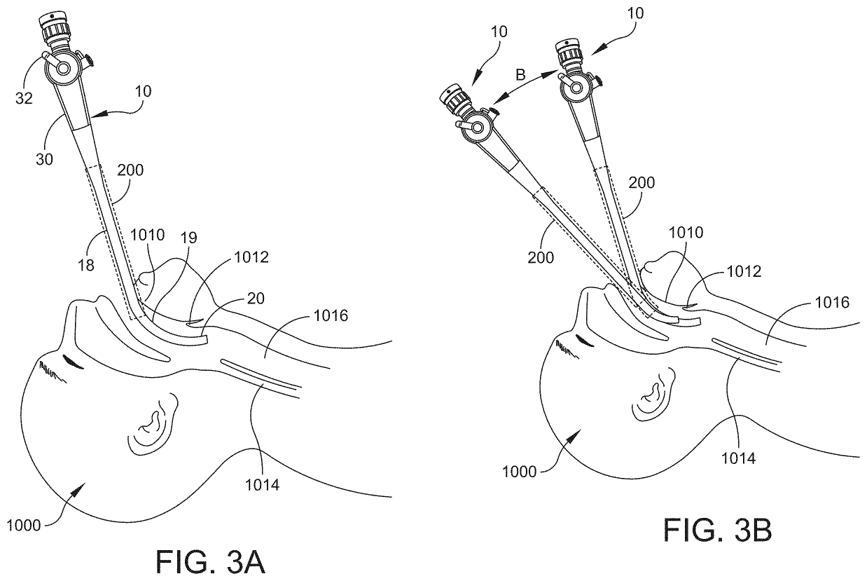

In one embodiment of the device, the rigid section 18 includes a curved portion 19. This curved portion 19 is adjacent to the flexible portion 20 of the sheath 12. This curved portion 19 is useful for lifting the tongue of the patient to open the airway when the patient is lying on his or her back. The curved portion 19 can engage the tongue of the patient so it can be lifted off of the posterior oropharynx, allowing better airway access. FIGS. 3A and 3B show an example of the orientation of the curved portion 19 in the sagittal plane view of a patient 1000. FIG. 3A shows a sagittal view of a patient 1000, showing the anatomical features such as the patient's tongue 1010, epiglottis 1012, esophagus 1014, and trachea 1016. FIGS. 3A and 3B show how the intubating device 10 can be rotated to along arrow B to different positions with respect to the patient 1000, while the flexible tip 20 remains in a position conducive to guiding an endotracheal tube 200 into position to maintain a patent airway. The endotracheal tube 200 is shown in broken lines in FIGS. 3A and 3B. It should be understood that FIG. 3B shows two positions of a single intubating device 10 for illustration purposes only. Only one intubating device 10 is needed at a given time. With this arrangement, the rigid section can be used to provide some lifting of the tongue and soft tissues. In addition, with this arrangement, the flexible tip provides for searching and steering toward of the patient airway (and vocal cords). With this arrangement, an airway can be intubated using the intubating device 10 without using a separate laryngoscope blade.

In one embodiment of the device, the rigid section of the sheath provides a mechanical advantage to the user when moving a patient's tongue out of the way to access an airway, while the flexible section allows the user to guide an endotracheal tube supported on the intubating device past the epiglottis and vocal cords of the patient with minimal or no injury to the patient. Because of the relative lengths of the rigid section and the flexible section of the sheath, the intubating device can be used without a laryngoscope blade, so there is decreased risk of excessive force applied by the operator of the intubating device to the patient's lower jaw and teeth. Because there is no need for a laryngoscope blade during insertion, the intubating device can be used with a single hand, enabling the second hand of the operator to be used for additional control if needed

Another aspect and advantage of the intubating device is unlike some of the prior art devices which require a flexible fiberoptic scope to passed through the vocal cords of a patient in to the distal part of the trachea to guide an ETT, which might cause trauma to the distal airway tissues, the proposed device does not have to be passed all the way into the airway of a patient. It can be passed slightly into the airway of the patient to provide a more direct guide. Alternatively, it can be disposed above the airway of a patient and guide (and watch) the ETT into the trachea.

FIG. 1A shows an endotracheal tube stop (endotracheal tube holder) 17 at the proximal end 16 of the sheath. The endotracheal tube stop 17 can be used to releasably secure, such as by friction, an endotracheal tube 200, or another airway securing device, to the sheath 12 of the intubating device. This allows a user to insert the intubating device and tube 10 into the body cavity of the user and then release the endotracheal tube 200 from the intubating device when the user is ready. When the endotracheal tube 200 is released from the intubating device 10, the intubating device 10 may be withdrawn from the endotracheal tube 200, leaving the endotracheal tube 200 in place in the airway of the patient.

In some embodiments, the endotracheal tube stop 17 is configured to frictionally secure a first end of the endotracheal tube 200 to the intubating device at the first end.

In the embodiment of FIG. 1A, the distal end 14 of the sheath 12 has a blunt tip, to avoid injury to the patient's anatomy as the intubating device is inserted into the body cavity of the patient. It is appreciated that other tips such a rounded, curved, or other shapes as is customary can be used.

In some embodiments of the intubating device, the imaging channel 26 is connected to an optical viewer, such as an eyepiece 34 or a display. FIG. 1A shows an eyepiece 34 located at a proximal end 15 of the endoscopic intubating device 10 on the control housing 30, so that a user can look through the eyepiece 34 to see the anatomy of the airway and to visually confirm placement of the endotracheal tube 200, or another airway securing device. The eyepiece 34 is preferably a high resolution eyepiece for conclusive placement of an endotracheal tube 200 within an airway of a patient. A diopter ring (not labeled) is provided for focusing.

Alternatively, the eyepiece 34 can be attached to a camera for display on a screen. Some fiberoptic embodiments of the device can have an eye piece. Some video scope embodiments of the device according to the present disclosure do not.

In some embodiments, the gas channel 28 is configured to be connected to at least one of a gas source and a suction source. Thus, the gas channel 28 can be used to provide gas to the body cavity when the gas channel 28 is connected to a gas source, and can be used to remove air, gas, or solids from the body cavity when the gas channel 28 is connected to a suction source.

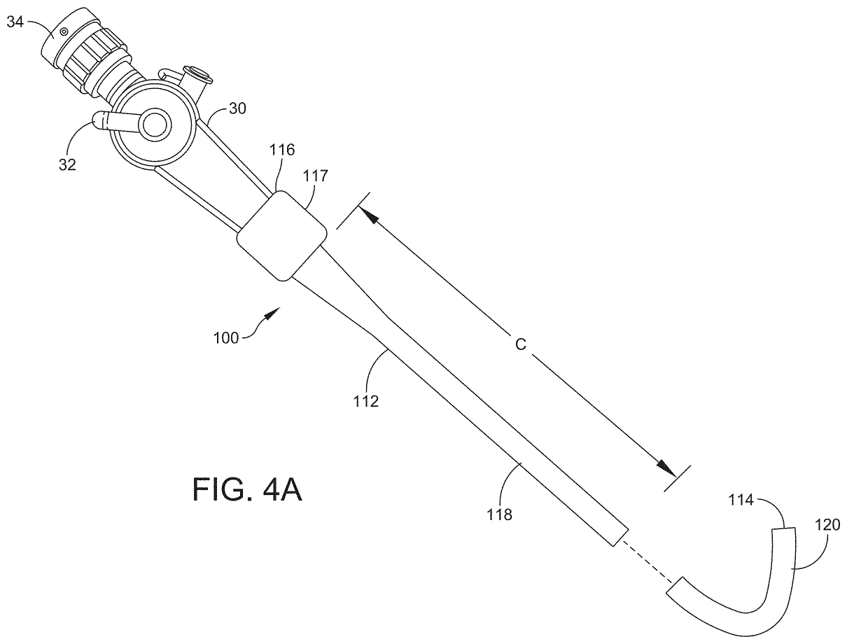

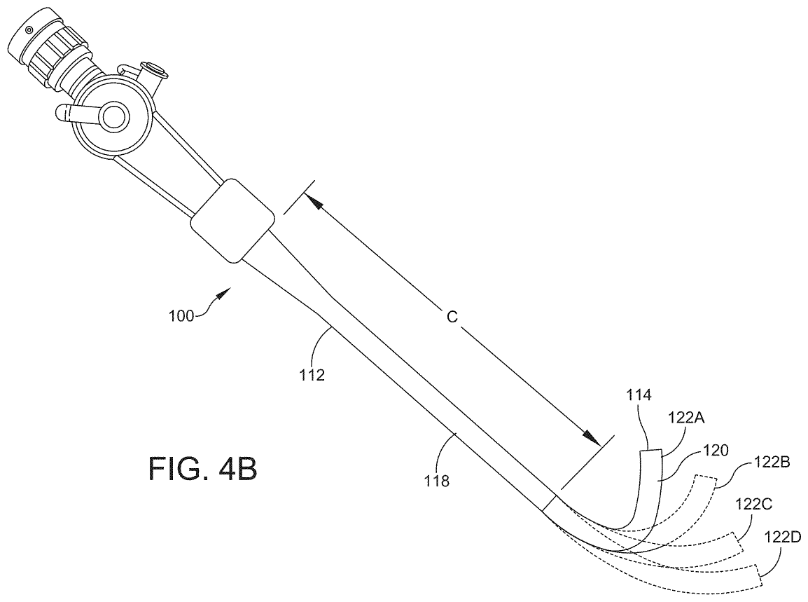

Turning to FIG. 4A, the embodiment of the endoscopic intubating device 100 of FIG. 4A is similar to the embodiment 10 of FIG. 1A, except the rigid section 118 of the sheath 112 does not include a curved portion towards the distal end 114 of the sheath 112. This embodiment is generally straight with a maneuverable portion 120.

The curvature of the rigid section is generally not adjustable within the body cavity of a patient or by use of the deflector 32.

FIG. 4A shows an endotracheal tube stop (endotracheal tube holder) 117 at the proximal end 116 of the sheath. The endotracheal tube stop 117 can be used to releasably secure, such as by friction, an endotracheal tube 200, or another airway securing device, to the sheath 112 of the intubating device. This allows a user to insert the intubating device 100 into the body cavity of the user and then release the endotracheal tube 200 from the intubating device when the user is ready. When the endotracheal tube 200 is released from the intubating device 100, the intubating device 100 may be withdrawn from the endotracheal tube 200, leaving the endotracheal tube 200 in place in the airway of the patient. Like the intubating device 10, the intubating device 100 includes a flexible section 120 that is selectively bendable, as shown in FIG. 4B, where the flexible section 120 has a curvature 122A that can be adjusted, such as to various curvatures 122B, 122C, 122D as shown in broken lines for illustration purposes in FIG. 4A. A user can adjust the curvature of the flexible section 120 of the sheath to navigate a body cavity, such as an airway of a patient.

In the embodiment of FIG. 4A, the rigid section 118 has a length C that is between 30 centimeters and 32 centimeters in length, and the flexible section 120 is between 3 centimeters and 5 centimeters in length when straightened.

In the embodiment of the intubating device 100 of FIG. 4A, the distal end 114 of the sheath 112 has a blunt tip, to avoid injury to the patient's anatomy as the intubating device is inserted into the body cavity of the patient. It is appreciated that other tips such a rounded, curved, or other shapes as is customary can be used.

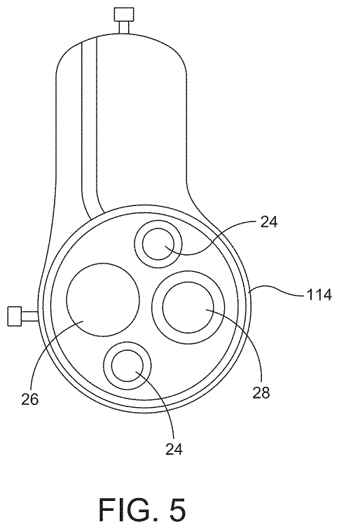

FIG. 5 shows an end view of the sheath 112, which is similar to the end view of the embodiment of FIG. 2. For the sake of brevity, the description of FIG. 2 applies to FIG. 5 and will not be repeated here.

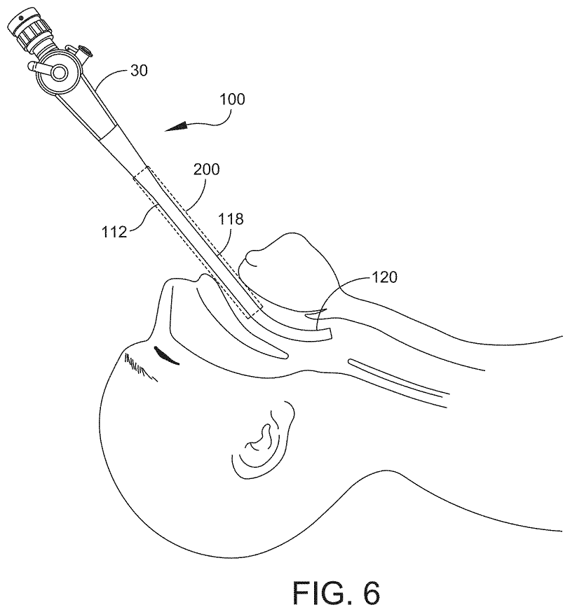

FIG. 6 shows the intubating device of FIG. 4A when it is being used to position the endotracheal tube 200 into the airway. For the sake of brevity, the descriptions of FIGS. 3A-3B apply to FIG. 6 and will not be repeated here.

FIGS. 7A-7C show various positions of the deflector (control lever) 32 that is rotatably secured to the control housing 30. In FIG. 7A, the deflector 32 is at a first position with respect to the housing 30, and the flexible section is straight. In FIG. 7B, the deflector 32 has been rotated counterclockwise from the first position to a second position with respect to the housing 30, and the flexible section 120 is flexed to the curvature shown. In FIG. 7C, the deflector 32 has been rotated clockwise from the first position to a third position with respect to the housing 30, and the flexible section 120 is extended. The deflector (or control lever) controls the flexible section at the distal end (tip) of the sheath. The deflector provides for movement of the flexible tip in a vertical plane, as shown in FIGS. 7A-7C.

In some embodiments, two wires extend from the handle of the device to the distal tip in the sheath. Rotating the deflector down changes the curvature of the flexible section to move the distal tip up and rotating the deflector up points the tip down. In some embodiments, side to side movement is accomplished by rotation of the body of the bronchoscope with the operator's wrist and shoulder. With this arrangement, each flexible guide wire is connected to the distal end of the sheath and is connected to the deflector (control lever). Rotation of the deflector 32 between the first position, the second position, and the third position of FIGS. 7A-7C causes the wire to be advanced or retracted in relation to the control housing 30, causing the respective curvatures of the flexible section 120 in FIGS. 7A-7C. Where two flexible wires are used, the deflector could cause a first wire to advance while causing a second wire to retract, with the first wire located at a first circumferential position along an inner perimeter of the sheath and with the second wire located at a diametrically opposed circumferential position along the inner perimeter of the sheath.

In some embodiments, each tip bending guide wire has a first end secured to the deflector 32 and movable by the deflector 32, and the guide wire has a second end secured to the distal end of the sheath. In some embodiments, rotation of the deflector 32 causes extension or retraction of each guide wire to effect a change in curvature of the flexible section. In some embodiments, each guide wire is contained within a guide wire channel extending through the rigid section 18 and the flexible section.

Other mechanisms can be used to cause the flexible section of the sheath to curve, without departing from the scope of the present disclosure.

FIGS. 8A-8D show some examples of how the flexible section 20 can by dynamically bent to adjust to anatomical features within a body cavity, such as an airway. In FIGS. 8A-8D, the flexible section 20 has different degrees of curvature to conform to the anatomy of an airway of a human patient or another patient.

It is also appreciated that the number and types of channels within the sheath 12, 112 can be changed without departing from the scope of the present disclosure.

It is also appreciated that the intubating device of the present disclosure may be used alone or in combination with other devices, such as laryngeal mask airway (LMA) devices.

It is also appreciated that the intubating device of the present disclosure is portable and reusable.

It is also appreciated that the intubating device of the present disclosure is easy to maintain and clean. For example, because there is no need for a laryngoscope blade, there is at least one less device to clean after use.

According to an aspect of the present disclosure, an intubating device for use with an endotracheal tube includes a sheath having a malleable but rigid first section and a steerable, flexible second section along the length of the sheath. The sheath is configured to be introduced into a body cavity, such as an airway of a patient. The steerable second section is located at a distal end of the sheath, and is connected to a distal end of the first section.

The first section is malleable, but is sufficiently rigid to facilitate placement of the second section in an airway. Before using the intubating device within a patient, a user, such as a medical practitioner, deforms the first section to a desired shape. For different procedures, different respective shapes of the first section may be desirable, because the first section of the sheath must pass through, deflect, lift, push, pull, and/or otherwise engage different tissue of a patient. Once the user deforms the malleable first section to the desired shape, the first section retains sufficient rigidity to allow the user to perform the intubating procedure. For example, in some embodiments, the first section retains sufficient rigidity that the user can engage the tongue and soft tissues of the patient with the rigid first section and pull on the first section to move the tongue and/or other soft tissue out of the airway of the patient. In some embodiments, the malleable first section is configured to at least substantially retain its shape during an endoscopic intubating procedure.

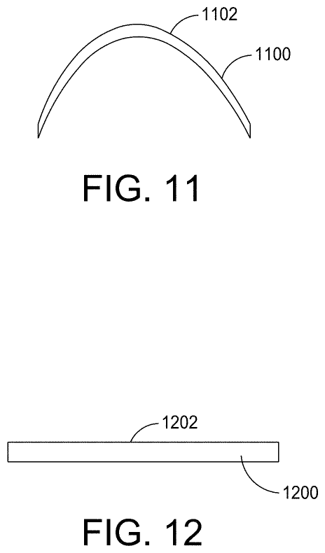

In some embodiments, the user can deform the malleable first section with a pipe bender, a shaping tool as shown in FIG. 11 or FIG. 12, or another device. In some embodiments, the user can deform the malleable first section by hand. The malleable first section retains its shape when no load is applied to the malleable first section. When the user holds the intubating device outside of the patient so the intubating device extends horizontally and so the sheath is cantilevered with the distal end of the sheath being a free end, the malleable first section retains its shape or at least substantially retains its shape.

In some embodiments, the first section comprises a stainless steel outer layer. The stainless steel outer layer may be bent by a user to a desired shape, and the stainless steel outer layer retains that shape or at least substantially retains that shape during an endoscopic procedure. When a new geometry of the stainless steel outer layer is desired during the same endoscopic procedure, or after the endoscopic procedure, a user may bend the stainless steel outer layer to a different desired shape.

In some embodiments, the first section comprises steel. In some embodiments, the first section comprises tungsten. The steel or tungsten portion of the first section is malleable, so that the first section can be bent to a desired shape, but so the first section is sufficiently rigid to retain the desired shape or at least substantially retain the desired shape during an endoscopic procedure, in which the intubating endoscopic device engages tissue of the patient or other structures of the patient, such as the tongue of the patient. In this way, the first section is useful for allowing the user to push aside tissue of the patient to place the second section of the sheath in a desired location within the patient

In comparison to the first section, which can be shaped by the user while the first section is outside of the patient, the second section is steerable by the user at all times, even when the second section is positioned within a cavity of a patient, such as an airway of a patient.

The second section of the sheath is at the distal end of the sheath. The second section is selectively bendable. For example, the second section can be selectively bent by use of one or more guide wires that extend within the sheath. The operation of the guide wires is described in relation to other embodiments of the present disclosure.

In some embodiments, a control housing is located at the proximal end of the sheath. In some embodiments, the control housing is positioned at another location. The control housing allows a user to control the curvature of the second section of the sheath. For example, the control housing allows a user to adjust guide wires within the sheath to curve the second section of the sheath to a desired curvature. In some embodiments, the control housing comprises a deflector for selectively bending the flexible section of the sheath. In some embodiments, the deflector is connected to one or more guide wires that are connected to the second section of the sheath.

Embodiments of an intubating device having a first section that is a malleable section provides various benefits. The intubating device includes the benefits of a shaft having a distal flexible tip discussed in relation to other embodiments of the present disclosure. Additionally, the first section of the shaft is malleable to allow further customization of the shaft. After altering the curvature of the first section of the shaft to a desired curvature, the first section of the shaft retains that desired curvature such that the first section is functionally rigid during its use, and the first section can be used to facilitate placement of the second section of the shaft in an airway of a patient.

A user may bend the first section of the sheath anywhere along the length of the first section based on the intubating procedure being performed. For example, a user could apply a slight bend near the distal end of the first section adjacent to the flexible second section to further facilitate placement of the first section around the base of the tongue. Another benefit is that the intubating device may be used during a nasal intubation by bending the shaft to fit the path from the nare to the oropharynx.

The malleable first section also allows a medical practitioner to adjust the shape of the sheath of the intubating device based on the particular anatomy of the patient. Because there are anatomical variations between different patients, a physician can pre-bend the malleable first section of the sheath to match a feature of a patient's airway, such as a curvature of a patient's airway. In some applications, a physician may apply a curvature to the malleable first section based on an imaging study of the patient, such as a magnetic resonance imaging (MRI) scan or a computed tomography (CT) scan of a patient's airway, which shows a curvature of the airway or other geometric features of the airway.

At the end of an intubating procedure, the medical practitioner may bend the malleable first section of the sheath to a different curvature, so that the sheath is suitable for use for a subsequent procedure, and/or is suitable for use in a different patient.

The intubating endoscopic device having a malleable first section of the sheath allows for greater flexibility by the medical practitioner.

The intubating endoscopic devices according to the present disclosure can be dimensioned and configured for use in pediatric procedures. Such embodiments of the endoscopic device are essentially the same as the endoscopic devices used for adult patients, but of smaller dimensions.

For example, in some embodiments of the intubating device for use as a pediatric intubating device, the sheath has a maximum external diameter of 2.5 millimeters and a total length of 24 centimeters. In some embodiments, the malleable first section has a length of 22 centimeters when the first section is straight and the second section has a length of two centimeters when the second section is straight. In some embodiments, the second section has a length in the range of about 2 centimeters to about 5 centimeters when the second section is straight.

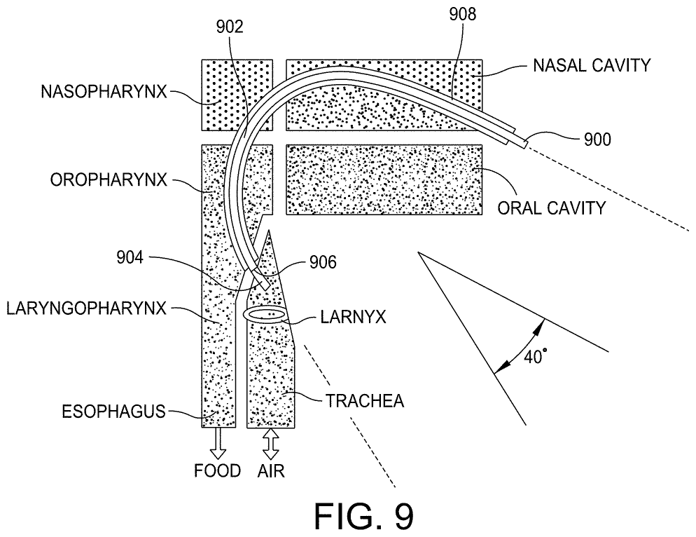

FIG. 9 shows a schematic sectional view of a patient with an embodiment of an intubating device 900 used for nasal intubation of the patient. The intubating device 900 has a sheath that includes a first section 902 that is malleable and a second section 904 at a distal end 906 of the first section 902 of the sheath. The first section 902 has been curved to allow the intubating device 900 to follow the curvature of the airway through the nasal cavity, the nasopharynx, and the oropharynx of the patient. The second section 904 extends into the larynx of the patient, and can be maneuvered within the larynx, for example by use of guide wires connected to the second section 904. An ETT 908 extends along the sheath over the outer surface of the first section 902. Once the ETT 908 is positioned in the airway, the intubating device 900 can be withdrawn from the airway.

The first section can be bent and also maintain its shape anywhere in a range of 0°-180°. As illustrated in the embodiment of FIG. 9, the first section 902 can be bent, for example, about 140° so as to the change the direction of the device by about 40° in order to pass the device through the nasal passage and into the naso- and oro-pharynx.

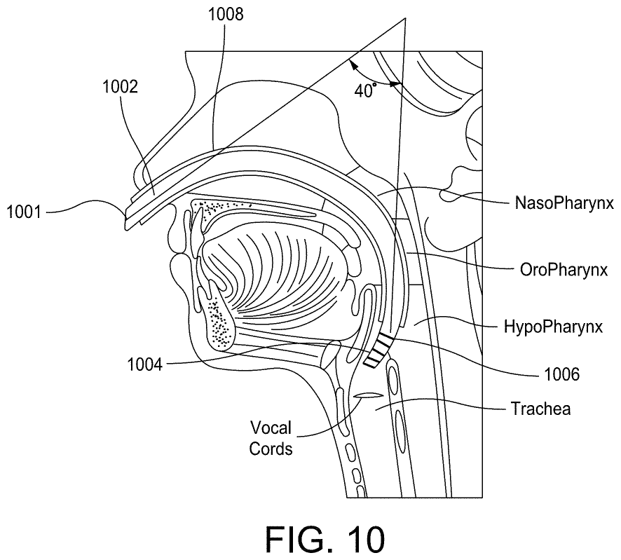

FIG. 10 shows a sectional view of a patient with an embodiment of an intubating device 1001 used for nasal intubation of the patient. The hybrid intubating device 1001 includes a shaft having a malleable but rigid first section 1002 and a flexible section 1004 extending from a distal end 1006 of the first section 1002. The first section 1002 has been bent to a curvature that allows the first section 1002 to extend through the nasal cavity and the pharyngeal passages. An ETT 1008 extends along the sheath over the outer surface of the first section 1002. FIG. 10 shows an acute angle, such as an angle of 40°, which may be the angle of axes extending from either end of the first section 1002 when the first section 1002 is bent.

Any of the features of any of the embodiments of the intubating device of the present disclosure and/or any of the dimensions of any of the embodiments of the intubating device of the present disclosure are applicable to any other embodiment of the intubating device of the present disclosure. For example, any of the intubating devices described herein may include a light source channel, an imaging channel, and a gas channel, or any other features. Each first section 902, 1002 may include any of the features of the rigid section 18 of the first embodiment, except the first section 902, 1002 is malleable. The second section 904, 1004 may include any of the features of the flexible section 20 of the first embodiment.

FIGS. 11 and 12 show embodiments of shaping tools that can be used to alter the shape of the first section. FIG. 11 shows a shaping tool 1100 that can be used to apply a curvature to the first section of a sheath. The first section of the sheath can be pressed against a surface 1102 of the shaping tool 1100 so that the malleable but rigid first section conforms to a profile of the shaping tool 1100. To straighten the malleable but rigid first section of the intubating device, a user may press the shaping tool 1200 against a surface 1202 of the malleable but rigid first section of the sheath of the intubating device so the first section of the sheath returns to a straight profile along its length.

In some embodiments, the shaping tool is configured to apply a curvature to the first section so the curvature of the first section 1002 allows the first section 1002 to extend through the nasal cavity and the pharyngeal passages of a patient.

In some embodiments, a shaping tool is configured as a U-shaped wall against which a sheath of an intubating device of the present disclosure can be pressed. In some embodiments, the U-shaped wall has a curved profile along its length, as shown in FIG. 11. In some embodiments, the U-shaped wall has a linear profile, as shown in FIG. 12. In some embodiments, the U-shaped cross section of the U-shaped wall is constant or at least substantially constant along the length of the shaping tool.

The shaping tool may be provided to a user as part of a kit that also includes an intubating device of the present disclosure.

In some embodiments, an intubating device of the present disclosure is dimensioned and configured to be used in pediatric intubating procedures. Such embodiments of the intubating device may have geometry that is similar to the geometry of any of the intubating devices shown or described in the present disclosure, but scaled down to fit in a child's airway.

Embodiments are not limited in their application to the details of construction and the arrangement of components set forth in the following description or illustrated in the drawings. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment, it is to be appreciated various alterations, modifications, and improvements will readily occur to those skilled in the art. Such alterations, modifications, and improvements are intended to be part of this disclosure, and are intended to be within the scope of the disclosure. Accordingly, the foregoing description and drawings are by way of example only.

What is claimed is:

1. An intubating device for use with an endotracheal tube, the intubating device comprising:
a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and
a control housing at the proximal end;
the control housing comprising a deflector for selectively bending the second section of the sheath.

2. The intubating device of claim 1, wherein the sheath is configured to be received within an endotracheal tube having an inner diameter of at least 6 mm.

3. The intubating device of claim 2, wherein the first section is 22 centimeters in length, and the second section is 2 centimeters in length.

4. The intubating device of claim 3, further comprising any or all of a light source channel, an imaging channel, and a gas channel enclosed by the sheath.

5. The intubating device of claim 1, further comprising at least one flexible wire extending along the length of the sheath connected to the deflector and to the second section.

6. The intubating device of claim 5, wherein the at least one flexible wire comprises two wires.

7. The intubating device of claim 1, wherein the first section is 22 centimeters in length, and the second section is 2 centimeters in length.

8. The intubating device of claim 1, wherein the first section comprises a curved portion towards the distal end adjacent to the second section.

9. The intubating device of claim 1, further comprising any or all of a light source channel, an imaging channel, and a gas channel enclosed by the sheath.

10. The intubating device of claim 9, wherein the imaging channel, the gas channel, and the light source channel terminate at the distal end of the sheath.

11. The intubating device of claim 9, wherein the imaging channel is configured to be connected to an optical viewer.

12. The intubating device of claim 9, wherein the gas channel is configured to be connected to at least one of a gas source and a suction source.

13. The intubating device of claim 9, further comprising a fluid channel.

14. The intubating device of claim 1, further comprising a tube stop configured to secure a first end of an endotracheal tube to the intubating device.

15. The intubating device of claim 1, further comprising at least one flexible wire extending along the length of the sheath connected to the deflector and to the second section.

16. The intubating device of claim 15, wherein the at least one flexible wire comprises two wires.

17. An intubating device for use with an endotracheal tube, the intubating device comprising:
a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and
the sheath extending along a light source channel, an imaging channel, and a gas channel;
a control housing at the proximal end;
the control housing comprising a deflector for selectively bending the second section of the sheath;
wherein the sheath is configured to be received within an endotracheal tube having an inner diameter of at least 6 mm; and
wherein the first section is between 30 centimeters and 32 centimeters in length, and the second section is between 3 centimeters and 5 centimeters in length.

18. An intubating kit for use with an endotracheal tube, the kit comprising:
a sheath having a distal end and a proximal end, the sheath being configured to be introduced into a body cavity, the sheath having a first section, and the sheath having a second section at the distal end, the first section being malleable and sufficiently rigid to facilitate placement of the second section in an airway, and the second section being selectively bendable; and
a control housing at the proximal end;
the control housing comprising a deflector for selectively bending the second section of the sheath; and
a shaping tool configured to apply a curvature to the first section of the intubating device, so the curvature of the first section allows the first section to extend through a nasal cavity and pharyngeal passages of a patient.

19. The kit of claim 18, wherein the first section of the intubating device is made of a first material having a first modulus of elasticity and the shaping tool is made of a second material having a second modulus of elasticity, the second material being than the first material, the first modulus of elasticity being greater than the second modulus of elasticity.

20. The kit of claim 19, wherein the shaping tool includes a U-shaped wall for applying the curvature of the U-shaped wall to the first section.
